(19) United States
(12) Patent Application Publication
(10) Pub. No.: US 2008/0294041 A1
(43) Pub. Date: Nov. 27, 2008

(54) Devices, Systems and Methods for Determining Sizes of Vessels

(76) Inventor: Ghassan S. Kassab, Zionsville, IN (US)

Correspondence Address: ICE MILLER LLP, ONE AMERICAN SQUARE, SUITE 3100, INDIANAPOLIS, IN, 46282-0200, US

(21) Appl. No.: 12/159,655

(22) PCT Filed: Jan. 25, 2007
(86) PCT No.: PCT/US07/01924

§ 371 Date: Jun. 30, 2008

Related U.S. Application Data
(60) Provisional application No. 60/761,783, filed on Jan. 25, 2006.

(51) Int. Cl. A61B 5/053; A61B 6/00

(52) U.S. Cl. 600/433; 600/547

(57) ABSTRACT

Devices, systems and methods are disclosed for determining the cross sectional area of a vessel. Through a combination of fluid injection with different conductivities and measurement of the resultant conductances, parallel tissue conductance measure is obtained that assists in determining the cross sectional area, taking into account the presence of a stent.

This patent application claims priority to U.S. Provisional Patent Application Ser. No. 60/761,783, filed Jan. 25, 2006; and is a continuation-in-part of U.S. patent application Ser. No. 11/063,836, filed Feb. 23, 2005, which is a continuation-in-part of U.S. patent application Ser. No. 10/782,149, filed Feb. 19, 2004; which claims priority to U.S. Provisional Patent Application Ser. No. 60/449,266, filed Feb. 21, 2003, and to U.S. Provisional Patent Application Ser. No. 60/493,145, filed Aug. 7, 2003, and to U.S. Provisional Patent Application Ser. No. 60/502,139, filed Sep. 11, 2003, the contents of each of which are hereby incorporated by reference in their entirety into this disclosure.

### BACKGROUND OF THE INVENTION

1. Field of the Invention

The present invention relates generally to medical diagnostics and treatment. More particularly, the present invention relates to devices, systems and methods for determining size of vessels, particularly in the presence of a stent.

2. Background of the Invention

The minimum cross-sectional area of a stented blood vessel is typically a good predictor of later events, e.g., restenosis. This observation has led to the notion of “bigger is better.” The limit to such larger size is, of course, vessel injury and dissection when the vessel is overly distended.

Angiography and intra-vascular ultrasound (IVUS) are two techniques that can determine the size of a vessel after stenting. A difficulty with the former is the poor resolution with the two dimensional (2-D) view typically obtained from a single x-ray projection. Furthermore, trapping of contrast agent near the stent lattice often creates hazing or shadows in the angiogram, which further reduces the accuracy of measurement. IVUS, on the other hand, tends to be more accurate and reliable. However, other factors limit its use. The cost of IVUS, the significant training required, and the subjectivity of image interpretation has significantly limited its usage to approximately 10% of routine procedures. Hence, it is desirable to introduce cheaper, easier and more objective tools for sizing of vessels after stenting.

### SUMMARY OF THE INVENTION

The present invention provides devices, systems and methods for determining the size of a blood vessel. The term “vessel,” as used herein, refers generally to any hollow, tubular, or luminal organ. Techniques according to the present invention are minimally invasive, accurate, reliable and easily reproducible.

In the prior parent applications, which all are incorporated by reference herein in their entirety, an impedance catheter was introduced that allows size determination of vessels based on electric impedance principle and a novel two-injection method. The previous devices, systems and methods did not disclose a technique of determining vessel size in the presence of a stent (typically a metal). In using prior embodiments, it is noted that contact of the impedance electrodes with the stent causes electrical shorting of signal and significant resulting noise, which prohibits accurate measurements. Furthermore, the presence of a metal in the measurement field also affects the conductivity. Thus, the present application proposes solutions to overcome these and other issues.

In one exemplary embodiment, the present invention is a device for determining a cross sectional size of a vessel. The device includes an elongated body having a longitudinal axis extending from a proximal end to a distal end, the body having a lumen along the longitudinal axis and enabling introduction of the distal end into a lumen of a vessel; a first excitation electrode and a second excitation electrode along the longitudinal axis, both located in respective grooves near the distal end; and a first detection electrode and a second detection electrode located in respective grooves along the longitudinal axis and in between the first and second excitation electrodes; wherein at least one of the first and second excitation electrodes is in communication with a current source, thereby enabling a supply of electrical current to the vessel, thereby enabling measurement of two or more conductance values in the blood vessel by the detection electrodes, and thereby enabling calculation of parallel tissue conductance in the vessel, whereby tissue conductance is the inverse of resistance to current flow, which depends on the cross sectional area of the blood vessel.

In another exemplary embodiment, the present invention is a device for determining a cross sectional area of a vessel. The device includes an elongated body having a lumen therethrough along its longitudinal length; a pair of excitation electrodes located in respective grooves on the elongated body; and a pair of detection electrodes located in respective grooves located in between the pair of excitation electrodes such that a distance between one detection electrode and its adjacent excitation electrode is equal to the distance between the other detection electrode and its adjacent excitation electrode; wherein at least one excitation electrode is in communication with a current source, thereby enabling a supply of electrical current to a lumen of a vessel, and enabling measurement of two or more conductance values at the lumen by the detection electrodes, resulting in an assessment of the cross sectional area of the blood vessel.

In another exemplary embodiment, the present invention is a catheter for determining a cross sectional area of a vessel. The device includes an elongated body having a lumen therethrough along its longitudinal length; a pair of excitation electrodes located in respective grooves on the elongated body; and a pair of detection electrodes located in respective grooves between the pair of excitation electrodes such that a distance between one detection electrode and its adjacent excitation electrode is equal to the distance between the other detection electrode and its adjacent excitation electrode; wherein when two solutions of differing conductive concentrations are introduced to a lumen of a vessel through the lumen of the elongated body at different times, two conductance measurements are made by the detection electrodes, resulting in a calculation of parallel tissue conductance at the lumen to determine cross sectional area.

In another exemplary embodiment, the present invention is a catheter for determining a cross sectional area of a vessel. The device includes an elongated body having a proximal end and a distal end and a lumen therethrough; a second body that terminates at the elongated body at a point between the proximal end and the distal end, and having a lumen that joins the lumen of the elongated body; a pair of excitation electrodes located in respective grooves at a distal end of the elongated body; and a pair of detection electrodes located in respective grooves between the pair of excitation electrodes; wherein when two solutions of differing conductive concentrations are introduced to a lumen of a blood vessel, located near the distal end of the elongated body, through the lumen of the second body, two conductance measurements are made by the detection electrodes, resulting in a calculation of parallel tissue conductance at the lumen to determine cross sectional area of the blood vessel.

In another exemplary embodiment, the present invention is a catheter system for determining a cross sectional area of a vessel as determined by resistance to flow of electrical currents through the lumen. The system includes an elongate wire having a longitudinal axis with a proximal end and a distal end; a catheter comprising an elongate tube extending from a proximal tube end to a distal tube end, the tube having a lumen and surrounding the wire coaxially; a first excitation electrode and a second excitation electrode each located in respective grooves along the longitudinal axis of the wire near the distal wire end; and a first detection electrode and a second detection electrode in respective grooves along the longitudinal axis of the wire and in between the first and second excitation electrodes, wherein at least one of the first and second excitation electrodes is in communication with a current source, thereby enabling a supply of electrical current to a lumen of a vessel, thereby enabling measurement of two or more conductance values at the lumen by the detection electrodes, and thereby enabling calculation of tissue conductance at the lumen, whereby tissue conductance is the inverse of resistance to current flow, which depends on the cross sectional area of the vessel.

In another exemplary embodiment, the present invention is a system for measuring cross sectional area of a blood vessel. The system includes a catheter assembly; a solution delivery source for injecting a solution through the catheter assembly and into a plaque site; a current source; and a data acquisition and processing system that receives conductance data from the catheter assembly and determines a cross sectional area of a lumen of a vessel, whereby the conductance is the inverse of resistance to current flow, which depends on the cross sectional area of the blood vessel.

In another exemplary embodiment, the present invention is a method for determining a cross sectional area of a vessel. The method includes introducing a catheter into a lumen of the vessel; providing electrical current flow to the lumen through the catheter; injecting a first solution of a first compound having a first concentration into the lumen; measuring a first conductance value at the plaque site; injecting a second solution of a second compound having a second concentration into the lumen, wherein the second concentration does not equal the first concentration; measuring a second conductance value at the lumen; and determining the cross sectional area of the vessel based on the first and second conductance values and the conductivity values of the first and second compounds.

### BRIEF DESCRIPTION OF THE DRAWINGS

FIG. 1 illustrates an impedance catheter according to an exemplary embodiment of the present invention in three magnifications wherein the four electrodes are spaced at the tip (two inner and two outer electrodes) in the top panel; a zoom of the embedded portion of the electrode arrangement is shown the middle panel; and a further zoom of the either circular or rectangular wire tunneling is shown in the lower panel.

FIG. 2 shows calibration of an impedance catheter in phantoms of saline (A) and in phantoms of saline with stent (B); and as shown, the slope remains similar but the intercept becomes non-zero for the stent (B).

FIG. 3 shows an exemplary measurement of vessel diameter in the presence of a stent according to an exemplary embodiment of the present invention.

### DETAILED DESCRIPTION OF THE INVENTION

This invention makes easy, accurate and reproducible measurements of the size of blood vessels within acceptable limits. This enables the determination of a blood vessel size with higher accuracy using basic techniques previously presented in more detail in the prior parent applications.

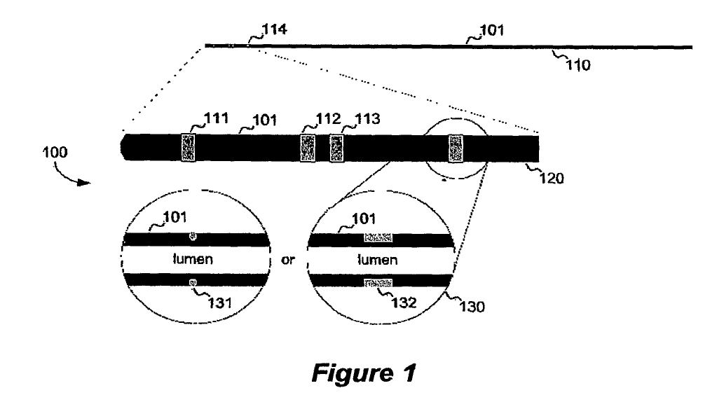

An exemplary embodiment of the present invention is presented as device 100 in FIG. 1. In this figure, a portion of a catheter 101 is presented at three different magnifications 110, 120 and 130. This catheter 101 has multiple electrodes 111, 112, 113 and 114 at one end. Such electrodes are used as described in the prior applications from which the present applications claims priority to. Thus, they will not be described in detail here. In brief, the two outer electrodes 111 and 114 are the excitation electrodes and the two inner electrodes 112 and 113 are the detection electrodes.

A further magnification 130 of the area around one of the electrodes 114 is presented. Multiple grooves or resting channels may be present in the body of catheter 101 to allow for the resting, cradling or supporting of the electrode therein. In one exemplary embodiment, the grooves 131 may be such that the electrode 114 is imbedded at least partially within the body of the catheter 101. In another exemplary embodiment, the groove or channel 132 may be in the form of a rectangular space such that the electrode 114 may rest therewithin. The grooves or channels may have other forms, which are also within the scope of the present invention.

More specifically, one of many advantages of the present invention is that its design provides for more accurate measurements. Previously, the four electrodes were exposed at the surface of the catheter where direct contact with stent was possible. In the present application, a design is proposed where grooves are made into the catheter such that the wires are made sub-surface. This design decreases surface contact of wires or electrodes with the stent while allowing the necessary exposure for the conducting electrode in the measurement field. Although two types of wire geometry (circular and rectangular) are shown, others are also possible and are within the scope of the present invention as long as at least some portion of each electrode is exposed to the interior of the blood vessel to enable measurement of electrical signals.

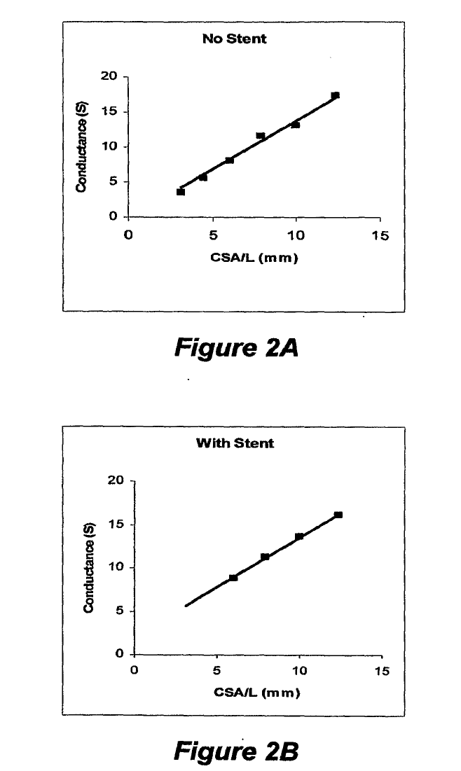

A second issue that is addressed by the novel design of the present invention is illustrated from experimental measurements. In the prior applications, it was shown that sizing (cross-sectional area, CSA) is related to the ratio of change in conductance to change in conductivity (slope of the conductivity-conductance relation). FIG. 2A shows the CSA/L-conductance relationship, which is expected to be linear with zero intercept. Based on the cylindrical model, and in the absence of a stent, the following relation is available:

$$G = \frac{C S A \cdot C}{L} \qquad [ 1 ]$$

where G is the conductance, current divided by voltage, C is the conductivity and L is the distance between the two inner electrodes. The slope of FIG. 2A corresponds to the conductivity C.

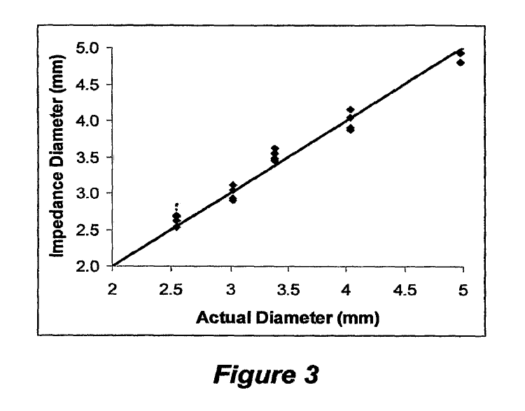

FIG. 2B shows the same relation in the presence of a stent. It is apparent from this finding that the slope of the curve remains unchanged but there is an offset that reflects the conductivity of the stent. A calibration of the specific stent (a number of different stent types are used in the art) reveals the offset and allows accurate sizing. Thus, FIG. 3 shows validation of the present approach where the stent was incorporated into the calibration. Several phantom tubes were measured and agreement is excellent.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A device for determining a cross sectional size of a vessel, the device comprising:
an elongated body having a longitudinal axis extending from a proximal end to a distal end, the body having a lumen along the longitudinal axis and enabling introduction of the distal end into a lumen of a vessel;
a first excitation electrode and a second excitation electrode along the longitudinal axis, both located in respective grooves near the distal end; and
a first detection electrode and a second detection electrode located in respective grooves along the longitudinal axis and in between the first and second excitation electrodes;
wherein at least one of the first and second excitation electrodes is in communication with a current source, thereby enabling a supply of electrical current to the vessel, thereby enabling measurement of two or more conductance values in the blood vessel by the detection electrodes, and thereby enabling calculation of parallel tissue conductance in the vessel, whereby tissue conductance is the inverse of resistance to current flow, which depends on the cross sectional area of the blood vessel.

2. The device of claim 1, wherein the conductance measurement takes into account an offset created in measured conductance by the presence of a stent in the cross sectional area that is being measured.

3. The device of claim 1, further comprising:
a data acquisition and processing system that receives conductance data from the detection electrodes and determines the conductance of the lumen.

4. The device of claim 1, further comprising:
a suction/infusion port located near the distal end, wherein said suction/infusion port is in communication with said lumen, thereby enabling injection of two or more solutions into the lumen.

5. The device of claim 4, wherein the solution comprises an NaCl solution.

6. The device of claim 4, wherein the lumen is in communication with a source of a solution to be injected therethrough and through the suction/infusion port into the lumen.

7. A device for determining a cross sectional area of a vessel, the device comprising:
an elongated body having a lumen therethrough along its longitudinal length;
a pair of excitation electrodes located in respective grooves on the elongated body; and
a pair of detection electrodes located in respective grooves located in between the pair of excitation electrodes such that a distance between one detection electrode and its adjacent excitation electrode is equal to the distance between the other detection electrode and its adjacent excitation electrode;
wherein at least one excitation electrode is in communication with a current source, thereby enabling a supply of electrical current to a lumen of a vessel, and enabling measurement of two or more conductance values at the lumen by the detection electrodes, resulting in an assessment of the cross sectional area of the blood vessel.

8. A catheter for determining a cross sectional area of a vessel, the device comprising:
an elongated body having a lumen therethrough along its longitudinal length;
a pair of excitation electrodes located in respective grooves on the elongated body; and
a pair of detection electrodes located in respective grooves between the pair of excitation electrodes such that a distance between one detection electrode and its adjacent excitation electrode is equal to the distance between the other detection electrode and its adjacent excitation electrode;
wherein when two solutions of differing conductive concentrations are introduced to a lumen of a vessel through the lumen of the elongated body at different times, two conductance measurements are made by the detection electrodes, resulting in a calculation of parallel tissue conductance at the lumen to determine cross sectional area.

9. The catheter of claim 8, wherein the detection and excitation electrodes have insulated electrical wire connections that run through the lumen of the elongated body.

10. The catheter of claim 8, wherein the detection and excitation electrodes have electrical wire connections that are embedded within the elongated body such that each wire is insulated from the other wires.

11. A catheter for determining a cross sectional area of a vessel, the device comprising:
an elongated body having a proximal end and a distal end and a lumen therethrough;
a second body that terminates at the elongated body at a point between the proximal end and the distal end, and having a lumen that joins the lumen of the elongated body;
a pair of excitation electrodes located in respective grooves at a distal end of the elongated body; and
a pair of detection electrodes located in respective grooves between the pair of excitation electrodes;
wherein when two solutions of differing conductive concentrations are introduced to a lumen of a blood vessel, located near the distal end of the elongated body, through the lumen of the second body, two conductance measurements are made by the detection electrodes, resulting in a calculation of parallel tissue conductance at the lumen to determine cross sectional area of the blood vessel.

12. The catheter of claim 11, wherein the detection and excitation electrodes have insulated electrical wire connections that run through the lumen and proximal end of the elongated body.

13. The catheter of claim 11, wherein the detection and excitation electrodes have electrical wire connections that are embedded within the elongated body such that each wire is insulated from the other wires.

14. The catheter of claim 11, further comprising a guide wire positioned through the proximal end of the elongated body, through the lumen of the elongated body and out of the distal end of the elongated body.

15. A catheter system for determining a cross sectional area of a vessel as determined by resistance to flow of electrical currents through the lumen, the system comprising:
an elongate wire having a longitudinal axis with a proximal end and a distal end;
a catheter comprising an elongate tube extending from a proximal tube end to a distal tube end, the tube having a lumen and surrounding the wire coaxially;
a first excitation electrode and a second excitation electrode each located in respective grooves along the longitudinal axis of the wire near the distal wire end; and
a first detection electrode and a second detection electrode in respective grooves along the longitudinal axis of the wire and in between the first and second excitation electrodes,
wherein at least one of the first and second excitation electrodes is in communication with a current source, thereby enabling a supply of electrical current to a lumen of a vessel, thereby enabling measurement of two or more conductance values at the lumen by the detection electrodes, and thereby enabling calculation of tissue conductance at the lumen, whereby tissue conductance is the inverse of resistance to current flow, which depends on the cross sectional area of the vessel.

16. The system of claim 15, wherein the wire comprises a pressure wire.

17. The system of claim 15, wherein the wire comprises a guide wire.

18. The system of claim 15, wherein the catheter comprises a guide catheter.

19. The system of claim 15, wherein the wire and the catheter are dimensioned so that a first solution can be infused through the tube lumen.

20. A system for measuring cross sectional area of a blood vessel, the system comprising:
a catheter assembly;
a solution delivery source for injecting a solution through the catheter assembly and into a plaque site;
a current source; and
a data acquisition and processing system that receives conductance data from the catheter assembly and determines a cross sectional area of a lumen of a vessel, whereby the conductance is the inverse of resistance to current flow, which depends on the cross sectional area of the blood vessel.

21. The system of claim 20, wherein the catheter assembly comprises:
an elongate wire having a longitudinal axis extending from a proximal wire end to a distal wire end;
a catheter comprising an elongate tube extending from a proximal tube end to a distal tube end, said tube having a lumen along its longitudinal axis, said tube surrounding the wire coaxially;
a first excitation impedance electrode and a second excitation impedance electrode each in respective grooves along the longitudinal axis of the wire, both located near the distal wire end; and
a first detection impedance electrode and a second detection impedance electrode each in respective grooves along the longitudinal axis of the wire, both located in between the first and second excitation electrodes.

22. A method for determining a cross sectional area of a vessel, the method comprising:
introducing a catheter into a lumen of the vessel;
providing electrical current flow to the lumen through the catheter;
injecting a first solution of a first compound having a first concentration into the lumen;
measuring a first conductance value at the plaque site;
injecting a second solution of a second compound having a second concentration into the lumen, wherein the second concentration does not equal the first concentration;
measuring a second conductance value at the lumen; and
determining the cross sectional area of the vessel based on the first and second conductance values and the conductivity values of the first and second compounds.
